(19) United States
(12) Patent Application Publication
(10) Pub. No.: US 2022/0409539 A1
(43) Pub. Date: Dec. 29, 2022

(54) PARTICLES FOR MULTI-DOSE DELIVERY

(71) Applicant: Fraunhofer USA Inc., Newark, DE (US)

(72) Inventors: Rikhav Praful Gala, Newark, DE (US); Robert Mark Jones, Springfield, PA (US); Konstantin Musiychuk, Hockessin, DE (US); Kevin Lawrence Stecca, Middletown, DE (US); Stephen John Streatfield, Kennett Square, PA (US)

(73) Assignee: Fraunhofer USA Inc., Newark, DE (US)

(21) Appl. No.: 17/778,505

(22) PCT Filed: Dec. 30, 2020
(86) PCT No.: PCT/US2020/067572

§ 371 Date: May 20, 2022

Related U.S. Application Data
(60) Provisional application No. 62/955,068, filed on Dec. 30, 2019.

(51) Int. Cl. A61K 9/16; A61K 39/39; A61K 39/07

(52) U.S. Cl. CPC A61K 9/167; A61K 39/39; A61K 39/07; A61K 2039/55577

(57) ABSTRACT

Particles offering multiple doses of an active agent to a subject via a single administration of the active agent to the subject are provided. The particles comprise multiple layers, including two dosing layers each having the active agent and an intervening layer that separates the two dosing layers, and provide a first dose of the active agent from the dosing layer covering the intervening layer upon exposure to an aqueous environment after the particles are administered to the subject, and later a second dose of the active agent from the dosing layer covered by the intervening layer after the intervening layer degrades in the body of the subject. Methods for preparing the particles are also provided. Methods for treating or preventing a disease or disorder in a subject in need thereof with two or more doses of an active agent via a single administration to the subject are further provided.

### CROSS-REFERENCE TO RELATED APPLICATION

This application claims priority to U.S. Provisional Application No. 62/955,068, filed Dec. 30, 2019, the contents of which are incorporated herein by reference in their entireties for all purposes.

### REFERENCE TO U.S. GOVERNMENT SUPPORT

This invention was made with government support under Grant No. HHSN272201200034C by the National Institute of Health. The United States has certain rights in the invention.

### FIELD OF THE INVENTION

This invention relates to particles providing multi-dose delivery of an active agent (e.g., active pharmaceutical ingredient (API)) to a subject via a single administration of the active agent and uses thereof.

### BACKGROUND OF THE INVENTION

Many pharmaceuticals, including vaccines, immunomodulators, antibodies and hormones require multiple doses to be administered to achieve the desired outcome in a patient or animal. Multiple administrations generally require the patient to repeatedly travel to a healthcare facility to have the pharmaceutical delivered by a trained professional, particularly if the route of administration is by injection. In addition to the medical personnel costs and inconvenience to the patient, repeated administrations also multiply the costs of materials used for delivery, such as needles and syringes. In addition, multiple administrations can be off-putting or even painful to a patient, which in some cases can lead to non-compliance with completing a full dosing regimen. To address these short comings, there is a need for a new method for delivering multiple doses of an active agent to a subject via a single administration.

### SUMMARY OF THE INVENTION

The present invention relates to novel particles comprising multiple layers for releasing an active agent in two or more doses in a subject after a single administration of the active agent to the subject. The inventors have surprisingly discovered multi-layered vaccine particles comprising a vaccine antigen in two or more dosing layers separated by one or more intervening layers for providing at least a bi-phasic release profile of the vaccine antigen.

A method for providing a subject in need thereof with at least two doses of an active agent after a single administration of the active agent to the subject is provided. The providing method comprises:

(a) administering a composition comprising particles to the subject, wherein the particles comprise the active agent and one or more biodegradable polymers, wherein each of the particles comprises a first dosing layer, a second dosing layer and a first intervening layer, wherein the first dosing layer covers the first intervening layer, the first intervening layer covers the second dosing layer, and the first intervening layer separates the first dosing layer from the second dosing layer, wherein the first and second dosing layers comprise at least 90 wt % of the active agent and the first intervening layer comprises at least 90 wt % of the one or more biodegradable polymers, wherein each of the first and the second dosing layers is soluble in an aqueous environment and the first intervening layer is hydrolytically degradable;

(b) providing a first dose of the active agent from the first dosing layer in the particles to the subject during a first dosing time period after step (a); and

(c) providing a second dose of the active agent from second dosing layer in the particles to the subject during a second dosing time period after step (b), wherein a first intervening time period separates the first dosing time period from the second dosing time period, whereby the subject receives at least two doses of the active agent after a single administration of the active agent to the subject. The subject may suffer a disease or disorder, and the active agent may be in an amount effective for treating or preventing the disease or disorder in the subject.

Each of the particles may further comprise a third dosing layer and a second intervening layer, the providing method may further comprise (d) providing a third dose of the active agent from the third dosing layer in the particles to the subject within a third dosing time period after step (c), wherein a second intervening time period separates the second dosing time period from the third dosing time period. The second dosing layer may cover the second intervening layer, the second intervening layer may cover the third dosing layer, and the second intervening layer may separate the second dosing layer from the third dosing layer. The first, second and third dosing layers may comprise at least 90 wt % of the active agent and the first and second intervening layers may comprise at least 90 wt % of the one or more biodegradable polymers. The third dosing layer may be soluble in an aqueous environment and the second intervening layer may be hydrolytically degradable.

According to the providing method, the subject may have first dosing, second dosing and first intervening average bodily fluid concentrations of the active agent during the first dosing, second dosing and first intervening time periods, respectively, and the first intervening average bodily fluid concentration may be no more than 50% of each of the first and second dosing average serum concentrations. The bodily fluid may be blood.

According to the providing method, the subject may have first dosing, second dosing, third dosing, first intervening and second intervening average bodily fluid concentrations of the active agent during the first dosing, second dosing, third dosing, first intervening and second intervening time periods, respectively, and each of the first and second intervening average bodily fluid concentrations may be no more than 50% of each of the first, second and third dosing average serum concentrations. The bodily fluid may be blood.

In one embodiment, the first intervening time period is longer than the first dosing time period. In another embodiment, the second dosing time period is longer than the first dosing time period.

According to the providing method, the particles may further comprise an excipient in the first dosing layer, the second dosing layer, or a combination thereof. The particles may be administered to the subject via oral, injectable, inhalational, sublingual, buccal, transdermal, rectal or vaginal delivery. The particles may further comprise an adjuvant in the first dosing layer, the second dosing layer or a combination thereof. The active agent may be a vaccine antigen, further comprising inducing an immune response to the antigen in the subject during the first and second dosing time periods. The induction of the immune response during the first dosing time period may extend into the intervening time period. The further induction of the immune response during the second dosing time period may extend beyond the second dosing time period. The immune response may be a T-cell response. The immune response may be a B-cell response.

A method for preparing three-layer particles for providing a subject in need thereof with at least two doses of an active agent after a single administration of the active agent to the subject is provided. The subject may suffer a disease or disorder, and the active agent may be in an amount effective for treating or preventing the disease or disorder in the subject.

In one embodiment, the three-layer or two-dose particle preparation method comprises:

(a) mixing a first aqueous solution with an organic phase, wherein the first aqueous solution comprises the active agent and the organic phase comprises one or more biodegradable polymers, whereby a primary emulsion of water-in-oil (W₁/O) is obtained;

(b) mixing the primary emulsion with a second aqueous solution, wherein the second aqueous solution comprises the active agent, whereby a double emulsion of water-in-oil-in-water (W₁/O/W₂) is obtained; and

(c) drying the double emulsion, whereby three-layer particles are obtained, wherein the three-layer particles comprise the active agent and the one or more biodegradable polymers, wherein each of the three-layer particles comprises a first dosing layer, a second dosing layer and a first intervening layer, wherein the first dosing layer covers the first intervening layer, the first intervening layer covers the second dosing layer, and the first intervening layer separates the first dosing layer from the second dosing layer, wherein the first and second dosing layers comprise at least 90 wt % of the active agent and the first intervening layer comprises at least 90 wt % of the one or more biodegradable polymers, wherein each of the first and the second dosing layers is soluble in an aqueous environment and the first intervening layer is hydrolytically degradable.

In another embodiment, the three-layer or two-dose particle preparation method comprises:

(a) mixing a first aqueous solution with an organic phase, wherein the first aqueous solution comprises the active agent and the organic phase comprises one or more biodegradable polymers, whereby a primary emulsion of water-in-oil (W₁/O) is obtained;

(b) mixing the primary emulsion with a second aqueous solution, wherein the second aqueous solution comprises the active agent, whereby a double emulsion of water-in-oil-in-water (W₁/O/W₂) is obtained;

(c) evaporating the organic phase in the double emulsion, whereby two-layer particles are obtained, wherein each of the two-layer particles comprises an intervening layer and a second dosing layer, the intervening layer covers the second dosing layer, the second dosing layer comprises the active agent, and the intervening layer comprises the one or more biodegradable polymers; and

(d) drying the two-layer particles within the second aqueous solution, whereby three-layer particles are obtained, wherein the three-layer particles comprise the active agent and the one or more biodegradable polymers, wherein each of the three-layer particles comprises a first dosing layer, a second dosing layer and a first intervening layer, wherein the first dosing layer covers the first intervening layer, the first intervening layer covers the second dosing layer, and the first intervening layer separates the first dosing layer from the second dosing layer, wherein the first and second dosing layers comprise at least 90 wt % of the active agent and the first intervening layer comprises at least 90 wt % of the one or more biodegradable polymers, wherein each of the first and the second dosing layers is soluble in an aqueous environment and the first intervening layer is hydrolytically degradable.

A method for preparing five-layer particles for providing a subject in need thereof with at least three doses of an active agent after a single administration of the active agent to the subject is also provided. The subject may suffer a disease or disorder, and the active agent may be in an amount effective for treating or preventing the disease or disorder in the subject.

In one embodiment, the five-layer or at least three-dose particle preparation method comprises:

(a) mixing a first aqueous solution with a first organic phase, wherein the first aqueous solution comprises the active agent and the first organic phase comprises one or more biodegradable polymers, whereby a primary emulsion of water-in-oil (W₁/O) is obtained;

(b) mixing the primary emulsion with a second aqueous solution, wherein the second aqueous solution comprises the active agent, whereby a double emulsion of water-in-oil-in-water (W₁/O/W₂) is obtained;

(c) drying the double emulsion, whereby three-layer particles are obtained, wherein each of the three-layer particles comprises a second dosing layer, a second intervening layer and a third dosing layer, wherein the second dosing layer covers the second intervening layer, the second intervening layer covers the third dosing layer, wherein the second and third dosing layers comprise the active agent and the second intervening layer comprises the one or more biodegradable polymers;

(d) mixing the three-layer particles with a third aqueous solution and a second organic phase, wherein the third aqueous solution comprises the active agent and the second organic phase comprises the one or more biodegradable polymers, whereby a mixture is prepared; and

(e) drying the mixture, whereby five-layer particles are obtained, wherein the five-layer particles comprise the active agent and the one or more biodegradable polymers, wherein each of the five-layer particles comprises a first dosing layer, a first intervening layer, the second dosing layer, the second intervening layer and the third dosing layer, wherein the first dosing layer covers the first intervening layer, the first intervening layer covers the second dosing layer, and the first intervening layer separates the first dosing layer from the second dosing layer, wherein the second dosing layer covers the second intervening layer, the second intervening layer covers the third dosing layer, and the second intervening layer separates the second dosing layer from the third dosing layer, wherein the first, second and third dosing layers comprise at least 90 wt % of the active agent and the first and second intervening layers comprise at least 90 wt % of the one or more biodegradable polymers, wherein each of the first, second and third dosing layers is soluble in an aqueous environment and each of the first and second intervening layers is hydrolytically degradable.

In another embodiment, the five-layer or at least three-dose particle preparation method comprises:

(a) mixing a first aqueous solution with a first organic phase, wherein the first aqueous solution comprises the active agent and the first organic phase comprises one or more biodegradable polymers, whereby a primary emulsion of water-in-oil (W₁/O) is obtained;

(b) mixing the primary emulsion with a second aqueous solution, wherein the second aqueous solution comprises the active agent, whereby a double emulsion of water-in-oil-in-water (W₁/O/W₂) is obtained;

(c) evaporating the organic phase in the double emulsion, whereby two-layer particles within the second aqueous solution are obtained, wherein each of the two-layer particles comprises a second intervening layer and a third dosing layer, the second intervening layer covers the third dosing layer, the third dosing layer comprises the active agent, and the second intervening layer comprises the one or more biodegradable polymers;

(d) drying the two-layer particles in the second aqueous solution, whereby three-layer particles are obtained, wherein each of the three-layer particles comprises a second dosing layer, the second intervening layer and the third dosing layer, the second dosing layer covers the second intervening layer and comprises the active agent;

(e) mixing the three-layer particles with a third aqueous solution and a second organic phase, wherein the third aqueous solution comprises the active agent and the second organic phase comprises the one or more biodegradable polymers, whereby a mixture is prepared;

(f) evaporating the second organic phase in the mixture, whereby four-layer particles within the third aqueous solution are obtained, wherein each of the four-layer particles comprises a first intervening layer, the second dosing layer, the second intervening layer and the third dosing layer, wherein the first intervening layer covers the second dosing layer and comprises the one or more biodegradable polymers; and (g) drying the four-layer particles within the third aqueous solution, whereby five-layer particles are obtained, wherein the five-layer particles comprise the active agent and the one or more biodegradable polymers, wherein each of the five-layer particles comprises a first dosing layer, the second dosing layer, the third dosing layer, the first intervening layer, and the second intervening layer, wherein the first dosing layer covers the first intervening layer, the first intervening layer covers the second dosing layer, and the first intervening layer separates the first dosing layer from the second dosing layer, wherein the second dosing layer covers the second intervening layer, the second intervening layer covers the third dosing layer, and the second intervening layer separates the second dosing layer from the third dosing layer, wherein the first, second and third dosing layers comprise at least 90 wt % of the active agent and the first and second intervening layers comprise at least 90 wt % of the one or more biodegradable polymers, wherein each of the first, second and third dosing layers is soluble in an aqueous environment and each of the first and second intervening layers is hydrolytically degradable.

In one embodiment, the first aqueous solution further comprises an excipient. In another embodiment, the second aqueous solution further comprises an excipient. In yet another embodiment, the third aqueous solution further comprises an excipient. In yet another embodiment, the particles further comprise an excipient in the first dosing layer, the second dosing layer, or a combination thereof.

In one embodiment, the first aqueous solution further comprises an adjuvant. In another embodiment, the second aqueous solution further comprises an adjuvant. In yet another embodiment, the third aqueous solution further comprises an adjuvant. In yet another embodiment, the particles further comprise an adjuvant in the first dosing layer, the second dosing layer, the third dosing layer, or a combination thereof.

The preparation method may further comprise storing the particles at a temperature of 20-40° C. for at least one year, wherein at least 80% of the activity of the active agent remains after storage.

According to the preparation method, the particles may further comprise an excipient in the first dosing layer, the second dosing layer, or a combination thereof. The particles may have an average particle size of 0.1-400 μm.

According to the preparation method, the active agent may be selected from the group consisting of a vaccine antigen, an antibody, an antibody-drug conjugate (ADC), a peptide, a protein, a pharmaceutical active drug molecule, an immuno-modulator, a hormone, a cell, and a combination thereof. The vaccine antigen may be selected from the group consisting of a protein, a polysaccharide, an epitope, a DNA, a RNA, a virus, a virus-like particle, and a combination thereof.

According to the preparation method, the one or more biodegradable polymers may be selected from the group consisting of poly (lactic-co-glycolic acid) (PLGA), poly (lactic acid) (PLA), polyethylene glycol (PEG), and combinations thereof. The first dosing layer may further comprise a cellulose-based polymer, a stabilizer, an antioxidant, a buffer, a bulking agent, or a combination thereof.

According to the preparation method, the second dosing layer may further comprise a cellulose-based polymer, a stabilizer, an antioxidant, a buffer, a bulking agent, or a combination thereof. The cellulose-based polymer may be selected from the group consisting of poly (vinyl alcohol) (PVA), ethyl cellulose (EC), methyl cellulose (MC), hydroxy propyl methyl cellulose (HPMC), hypromellose acetate succinate (HPMCAS), alginate, chitosan, and a combination thereof. The stabilizer may be selected from the group consisting of a carbohydrate, a hydrolyzed gelatin, a surfactant, a buffer salt, a culture medium, an antibiotic, and a combination thereof. The carbohydrate may be selected from the group consisting of sucrose, trehalose, lactose, dextrose, galactose, maltose, maltodextrin, dextrin, glycogen, starch, and a combination thereof. The surfactant may be selected from the group consisting of polysorbate 80, sorbitan monooleate, sorbitan laurate, sorbitan stearate, sorbitan tristearate, sorbitan sesquioleate, sorbitane trioleate, sorbitan monopalmitate, polyoxyethylenesorbitan monopalmitate, polyethylene glycol, sorbitan monostearate, polyoxyethylenesorbitan trioleate, polyoxyethylenesorbitan tristearate, D-tocopherol, and combinations thereof.

According to the preparation method, the active agent may be a vaccine antigen and the particles may further comprise an adjuvant.

Particles prepared according to any one of the preparation methods of the present invention are provided. The particles are dry.

Particles for providing a subject in need thereof with at least two doses of an active agent after a single administration of the active gent to the subject are provided. The particles comprise the active agent and one or more biodegradable polymers. Each of the particles comprises a first dosing layer, a second dosing layer and a first intervening layer. The first dosing layer covers the first intervening layer, the first intervening layer covers the second dosing layer, and the first intervening layer separates the first dosing layer from the second dosing layer. The first and second dosing layers comprise at least 90 wt % of the active agent and the first intervening layer comprises at least 90 wt % of the one or more biodegradable polymers. Each of the first and the second dosing layers is soluble in an aqueous environment and the first intervening layer is hydrolytically degradable. The subject may suffer a disease or disorder, and the active agent may be in an amount effective for treating or preventing the disease or disorder in the subject.

The particles of the present invention may have an average particle size of 0.1-400 μm.

In the particles, the active reagent may be selected from the group consisting of a vaccine antigen, an antibody, an antibody-drug conjugate (ADC), a peptide, a protein, a pharmaceutical active drug molecule, an immuno-modulator, a hormone, a cell, and a combination thereof. The vaccine antigen may be selected from the group consisting of a protein, a polysaccharide, an epitope, a DNA, a RNA, a virus, a virus-like particle, and a combination thereof. The one or more biodegradable polymers may be selected from the group consisting of poly (lactic-co-glycolic acid) (PLGA), poly (lactic acid) (PLA), and combinations thereof. Where the active agent is a vaccine antigen, the particles may further comprise an adjuvant.

In the particles, the first dosing layer may further comprise a cellulose-based polymer, a stabilizer, an antioxidant, a buffer, a bulking agent, or a combination thereof. The second dosing layer may further comprise a cellulose-based polymer, a stabilizer, an antioxidant, a buffer, a bulking agent, or a combination thereof. The cellulose-based polymer may be selected from the group consisting of poly (vinyl alcohol) (PVA), ethyl cellulose (EC), methyl cellulose (MC), hydroxy propyl methyl cellulose (HPMC), hypromellose acetate succinate (HPMCAS), alginate, chitosan, and a combination thereof. The stabilizer may be selected from the group consisting of a carbohydrate, a hydrolyzed gelatin, a surfactant, a buffer salt, a culture medium, an antibiotic, and a combination thereof. The carbohydrate may be selected from the group consisting of sucrose, trehalose, lactose, dextrose, galactose, maltose, maltodextrin, dextrin, glycogen, starch, and a combination thereof. The surfactant may be selected from the group consisting of polysorbate 80, sorbitan monooleate, sorbitan laurate, sorbitan stearate, sorbitan tristearate, sorbitan sesquioleate, sorbitane trioleate, sorbitan monopalmitate, polyoxyethylenesorbitan monopalmitate, polyethylene glycol, sorbitan monostearate, polyoxyethylenesorbitan trioleate, polyoxyethylenesorbitan tristearate, D-tocopherol, and combinations thereof.

A composition is provided. The composition comprises the particles of the present invention and a pharmaceutically acceptable excipient. The composition may be in an oral, injectable, inhalational, sublingual, buccal, transdermal, rectal or vaginal delivery form. The composition may be in the form of capsules, tablets, granules or suspensions of particles for oral delivery to gastrointestinal (GI) tract. The composition may be in the form of an orally dissolvable film (ODF) for buccal or sublingual delivery. The composition may be in the form of a transdermal patch for transdermal delivery. The composition may be in the form of a cream, ointment or gel for topical delivery. The composition may be in the form of a hydrogel matrix for subcutaneous or intramuscular injection. The composition may be in an injectable form for intravenous, subcutaneous or intramuscular delivery.

### BRIEF DESCRIPTION OF THE DRAWINGS

FIG. 1 shows a schematic diagram of a multi-layered particle for delivery of two doses of an active pharmaceutical ingredient (API). The layers containing the API (i.e., outer layer and inner layer) are shown in green while the intervening layer (i.e., middle layer) containing at least one biodegradable polymer is shown in yellow.

FIG. 2 shows a desirable antigen release profile from multi-layered vaccine particles.

FIG. 3 shows scanning electron microscope images of a spray dried particles containing vaccine antigen PA83 in core without coating on top of the core.

FIG. 4 shows Western blot analysis of spray dried particles containing vaccine antigen PA83 in core without coating on top of the core, indicating the presence and stability of the PA83 in formulations F9 and F10 after storage at 25° C.±3° C. for a 6-month period. Lane 1: Molecular ladder; Lane 2: F9 6-month sample; Lane 3: F9 3-month sample; Lane 4: F9 0-day (fresh) sample; Lane 5: Blank; Lane 6: F10 6-month sample; Lane 7: F10 0-day (fresh) sample; Lane 8: Blank; Lanes 9-12: Standards.

FIG. 5 shows Western blot analysis of multi-layered vaccine particles indicating the presence of vaccine antigen PA83 in different layers of the particles. Lane 1: Molecular weight ladder; Lane 2: the total vaccine antigen content present in all layers of the particles; Lane 3: vaccine antigen released following dissolution of the particles in PBS, corresponding to the antigen in the outermost layer, which provides the initial burst release, i.e., first dose; Lane 4: vaccine antigen released from the middle, polymeric, layer following incubation for 1 hour in PBS; Lane 5: vaccine antigen in the inner most layer, or core, that can be released at a later time to provide a second dose of the antigen. The antigen from the core was released from the polymer with the addition of SDS sample buffer and heating at 95° C. for 5 minutes.

FIG. 6 shows antigen specific antibody titers of immunized mice. Animals were immunized IM with vaccine antigen PA83 twice (group 2, squares) (5 μg on day 0 and 5 μg on day 21) or once (group 4, diamonds) (5 μg on day 0), or once (5 μg on day 0) with PLGA-PA83 (i.e., PA83 within a PLGA coating) with additional soluble PA83 as the prime dose (group 3, circles) (5 μg on day 0). A control group of mice received saline twice (triangles) (day 0 and day 21). A QS21 based adjuvant was included for the prime dose on day 0 in all groups. The group receiving a single dose of PLGA-PA83 plus soluble PA83 (group 3, circles) showed comparable anti-PA IgG to mice receiving two doses of soluble PA83 (group 2, squares).

FIG. 7 shows Toxin Neutralization Activity of antibodies generated by immunized mice in an immunogenicity study. The group receiving a single dose of PLGA-PA83 plus soluble PA83 (group 3) showed comparable Toxin Neutralizing Activity (TNA) to mice receiving two doses of soluble PA83 (group 2).

### DETAILED DESCRIPTION OF THE INVENTION

The present invention provides multi-layered particles offering at least two distinctive phases of dosing of an active agent such as an active pharmaceutical ingredient (API) in a subject after a single administration. The invention was made based on inventors' surprising discovery of a novel, vaccine stabilization/administration approach based on layering of spray-dried pharmaceuticals with biodegradable polymers to create a multiple-dose formulation in the form of particles that can be delivered as a single administration. The multi-layered particles may be prepared and used for treating or preventing a disease or disorder in a subject, especially when multiple doses of the same active agent are required.

According to the present invention, each particle comprises multiple layers and an active agent such as an API in at least two dosing layers separated by an intervening layer such that the active agent is released in at least two distinct phases of dosing from the particle. The terms “layer” and “coating” are used herein interchangeably and refer to a continuous and enclosing sheet structure having homogenous ingredients. The multiple layers or coatings in a particle enclose or cover one by one from the outmost surface to the core of the particle.

The term “dosing layer” as used herein refers to a layer in a particle that comprises an active agent such as an API, whose release from the dosing layer provides a dose of the active agent during a dosing time period after the dosing layer is exposed to an aqueous environment, for example, an aqueous solution and becomes soluble. The dosing layer may further comprise a cellulose-based polymer, a stabilizer, an antioxidant, a buffer, a bulking agent, or a combination thereof

The term “intervening layer” as used herein refers to a layer in a particle that comprises at least one biodegradable polymer and no or a very small amount of an active agent such as an API, for example, at no more than 10 wt %, 5 wt % or 1 wt % of the total weight of the API in the particle. The biodegradable polymer may be selected from the group consisting of poly (lactic-co-glycolic acid) (PLGA), poly (lactic acid) (PLA) and combinations thereof. The intervening layer degrades hydrolytically over time when exposed to an aqueous environment. An intervening layer protects an inner dosing layer enclosed or covered by the intervening layer from being exposed to an aqueous environment and therefore prohibits releasing the active agent from the inner dosing layer until the biodegradable polymer in the intervening layer degrades.

When the intervening layer separates two dosing layers in a particle, an outermost dosing layer that encloses or covers the intervening layer and an inner dosing layer enclosed or covered by the intervening layer, the outermost dosing layer releases an active agent as a first dose upon exposure of the particle to an aqueous environment while the inner dosing layer does not release the active agent as a second dose until the biodegradable polymer in the intervening layer degrades and the inner dosing layer is exposed to the aqueous environment. The time gap, i.e., an intervening time period, between the first and the second doses of the active agent may be controlled by modifying the intervening layer, for example, its thickness and ingredients (e.g., biodegradable polymers). The outermost dosing layer may release its active agent when the particle is reconstituted in an aqueous solution prior to administration.

Various factors affect the release of the active agent (e.g., antigen). Polymer properties influence the release profile of the encapsulated active agent (e.g., antigen). The release of the antigen may be controlled by varying the molar ratios of individual monomers (which form the polymer); changing the molecular weight of the monomers, and/or chemical modifications of the monomers by changing the functional group. The quantity of the polymer used (e.g., 1-30% w/w) to make the intervening layer may also alter the release of the encapsulated antigen as well. To achieve a desirable release profile for the encapsulated active agent, conventional techniques may be used to modify the properties of the intervening biodegradable polymer layer, for example, adjusting molecular weight of the polymer or functional groups on the polymer.

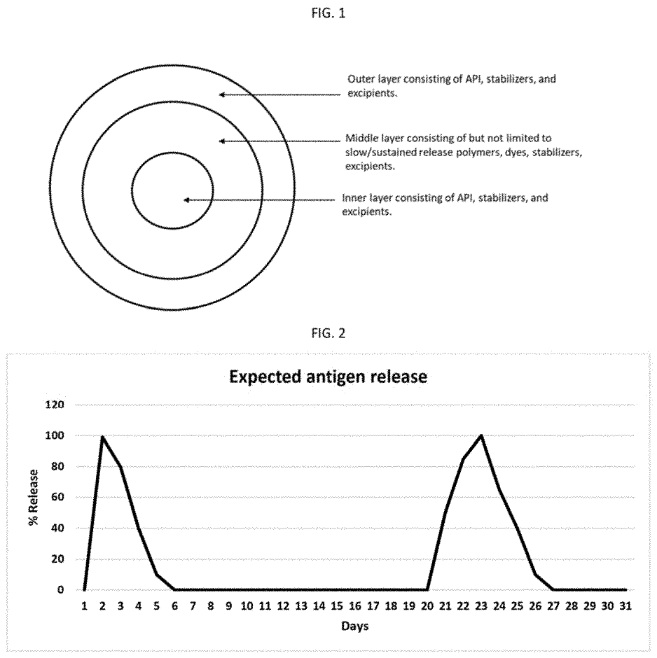

FIG. 1 illustrates a simplest case of a multi-layered particle for delivering two doses of an API. The particle contains three layers: an outer layer enclosing or covering a middle layer, which encloses or covers an inner layer. The outer layer and the inner layer are dosing layers each consisting of an AIP, stabilizers and excipients while the middle layer is an intervening layer comprising slow/sustained release polymers, dyes, stabilizers and excipients. The slow/sustained release polymers are biodegradable polymers. The innovative layering of the API and polymer coatings generates particles having an average diameter of 0.1-400 μm and offers distinct phases of dosing from a single administration.

In one embodiment, an outermost dosing layer in a particle is soluble when reconstituted in an aqueous solute prior to administration or when directly administered and provides the first dose of an active agent (e.g., API). The biocompatible/biodegradable polymers in an intervening layer provide a barrier to immediate release of the active agent from an inner dosing layer and facilitate controlled release of the active agent in an inner dosing layer. The outermost dosing layer comprising the active agent is in a form that is easily solubilized in an aqueous environment such that the active agent from the outermost dosing layer becomes available as a first dose of the active agent to the body of a subject immediately after administration of the particle to the subject, whereas the inner core or inner dosing layer, also comprising the active agent in a form that is easily solubilized in an aqueous environment, is shielded by the intervening layer comprising biodegradable polymers, is not immediately available after administration and is released at a later time once the middle polymeric intervening layer degrades or is disrupted in the body, thus providing a second dose of the active agent.

In another embodiment, multi-layered particles such as those depicted in FIG. 1 are used for delivering a vaccine antigen. The desirable antigen release profile is bi-phasic. The first or initial release or dosing phase would be immediate at the time of injection (Day 0). Following the initial dose, there would ideally be an intervening time period of no release for approximately 7-30 days, or up to 2, 3, 6 or 12 months. During this intervening time period, the polymeric coated particles undergo surface erosion finally leading to sustained or controlled release of the antigen from the core during the second release phase. This second release phase would ideally begin approximately 7-30 days, or up to 2, 3, 6 or 12 months, after administration and would last anywhere from 1 day to 1 month. This bi-phasic release of the antigen from the single administration is shown in FIG. 2 and is beneficial for antigens which need multiple doses at regular intervals (prime and boost doses). The multi-layered particles act as a depot of the antigen in the body, followed by pulsed release to stimulate the immune system and confer lasting immunity. Additional layers could be included to give multiple pulses.

Particles for providing at least two doses of an active agent to a subject in need thereof after a single administration of the active agent to the subject are provided. The at least two-dose particles comprise the active agent and one or more biodegradable polymers. Each at least two-dose particle comprises a first dosing layer, a second dosing layer and a first intervening layer. The first dosing layer covers the first intervening layer. The first intervening layer covers the second dosing layer. The first intervening layer separates the first dosing layer from the second dosing layer. The first and second dosing layers comprise at least 80 wt %, 85 wt %, 90 wt %, 95 wt %, 99 wt % or 100 wt % of the active agent. The first intervening layer comprises at least 80 wt %, 85 wt %, 90 wt %, 95 wt %, 99 wt % or 100 wt % of the one or more biodegradable polymers. The first and the second dosing layers are soluble in an aqueous environment. The first intervening layer is hydrolytically degradable. The subject may suffer a disease or disorder, and the active agent in the at least two doses may be in an amount effective for treating or preventing the disease or disorder in the subject.

The particles may comprise one or more additional dosing layers and/or one or more additional intervening dosing layers to provide one or more additional doses of the active agent as long as at least one intervening dosing layer separates two dosing layers.

For example, the particles may provide at least three doses of an active agent to a subject in need thereof after a single administration of the active agent to the subject. Such at least three-dose particles comprise the active agent and the one or more biodegradable polymers. Each at least three-dose particle comprises a first dosing layer, a second dosing layer, a third dosing layer, a first intervening layer and a second intervening layer. The first dosing layer covers the first intervening layer, the first intervening layer covers the second dosing layer, and the first intervening layer separates the first dosing layer from the second dosing layer. The second dosing layer covers the second intervening layer, the second intervening layer covers the third dosing layer, and the second intervening layer separates the second dosing layer from the third dosing layer. The first, second and third dosing layers may comprise at least 80 wt %, 85 wt %, 90 wt %, 95 wt %, 99 wt % or 100 wt % of the active agent. The first and second intervening layers may comprise at least 80 wt %, 85 wt %, 90 wt %, 95 wt %, 99 wt % or 100 wt % of the one or more biodegradable polymers. Each of the first, second and third dosing layers is soluble in an aqueous environment. Each of the first and second intervening layers is hydrolytically degradable. The subject may suffer a disease or disorder, and the active agent in the at least three doses may be in an amount effective for treating or preventing the disease or disorder in the subject.

The particles are dry. The three-layer or at least two-dose particles are dry. The five-layer or at least three-dose particles are dry. The particles may have an average particle size of 0.01-1,000 μm, 0.1-500 μm 0.1-400 μm, 10-400 μm, 10-100 μm or 15-100 μm. At least 70%, 75%, 80%, 85%, 90%, 95%, 99% or 100% of the particles may have a particle size of 0.01-1,000 μm, 0.1-500 μm 0.1-400 μm, 10-400 μm, 10-100 μm or 15-100 μm.

The active reagent in the particles may be an active pharmaceutical ingredient (API) for treating or preventing a disease or disorder in a subject. In one embodiment, the active agent is selected from the group consisting of a vaccine antigen, an antibody, an antibody-drug conjugate (ADC), a peptide, a protein, a pharmaceutical active drug molecule, an immuno-modulator, a hormone, a cell and a combination thereof. The vaccine antigen may be selected from the group consisting of a protein, a polysaccharide, an epitope, a DNA, a RNA, a virus (e.g., attenuated virus or engineered virus), a virus-like particle, and a combination thereof. Where the active agent is a vaccine antigen, the particles may further comprise an adjuvant.

The biodegradable polymers in the particles may be selected from the group consisting of poly (lactic-co-glycolic acid) (PLGA), which may have a ratio of lactic acid to glycolic acid ranging from 1:9 to 9:1, poly (lactic acid) (PLA) and combinations thereof.

The first dosing layer in the particles may further comprise a cellulose-based polymer, a stabilizer, an antioxidant, a buffer, a bulking agent, or a combination thereof. The second dosing layer in the particles may further comprise a cellulose-based polymer, a stabilizer, an antioxidant, a buffer, a bulking agent, or a combination thereof. The cellulose-based polymer may be selected from the group consisting of poly (vinyl alcohol) (PVA), ethyl cellulose (EC), methyl cellulose (MC), hydroxy propyl methyl cellulose (HPMC), hypromellose acetate succinate (HPMCAS), alginate, chitosan and a combination thereof. The chitosan may be glycol chitosan, EDTA-chitosan or N-trimethyl chitosan. The stabilizer may be selected from the group consisting of a carbohydrate, a hydrolyzed gelatin, a surfactant, a buffer salt, a culture medium, an antibiotic and a combination thereof. The carbohydrate may be selected from the group consisting of sucrose, trehalose, lactose, dextrose, galactose, maltose, maltodextrin, dextrin, glycogen, starch, and a combination thereof. The surfactant may be selected from the group consisting of polysorbate 80, sorbitan monooleate, sorbitan laurate, sorbitan stearate, sorbitan tristearate, sorbitan sesquioleate, sorbitane trioleate, sorbitan monopalmitate, polyoxyethylenesorbitan monopalmitate, polyethylene glycol, sorbitan monostearate, polyoxyethylenesorbitan trioleate, polyoxyethylenesorbitan tristearate, D-tocopherol and combinations thereof.

A method for preparing the particles of the present invention is provided. The particles may provide at least two or three doses of an active agent to a subject after a single administration of the active agent to the subject.

In one embodiment, for the at least two-dose or three-layer particles, the preparation method comprises mixing a first aqueous solution with an organic phase to obtain a primary emulsion of water-in-oil (W₁/O). The first aqueous solution comprises the active agent and the organic phase comprises one or more biodegradable polymers. The primary emulsion is then mixed with a second aqueous solution to make a double emulsion of water-in-oil-in-water (W₁/O/W₂). The second aqueous solution comprises the active agent. The double emulsion is dried. As a result, three-layer particles are obtained. In the resulting three-layer particles, the first dosing layer may be derived from the second aqueous solution, the second dosing layer may be derived from the first aqueous solution, and the first intervening layer derived from the organic phase.

In another embodiment, for the at least two-dose or three-layer particles, the preparation method comprises mixing a first aqueous solution with an organic phase to make a primary emulsion of water-in-oil (W₁/O), where the first aqueous solution comprises an active agent and the organic phase comprises one or more biodegradable polymers; mixing the primary emulsion with a second aqueous solution, which comprises the active agent, to make a double emulsion of water-in-oil-in-water (W₁/O/W₂); and drying (e.g., spray drying and lyophilizing) the two-layer particles within the second aqueous solution. The organic phase in the double emulsion may be evaporated to obtain two-layer particles within the second aqueous solution such that each of the two-layer particles comprises an intervening layer and a second dosing layer, the intervening layer covers the second dosing layer, the second dosing layer comprises the active agent, and the intervening layer comprises the one or more biodegradable polymers. As a result, three-layer particles are obtained. In the resulting three-layer particles, the first dosing layer may be derived from the second aqueous solution, the second dosing layer may be derived from the first aqueous solution, and the first intervening layer derived from the organic phase.

In one embodiment, for the at least three-dose or five-layer particles, the preparation method comprises mixing a first aqueous solution with a first organic phase to obtain a primary emulsion of water-in-oil (W₁/O). The first aqueous solution comprises the active agent and the first organic phase comprises one or more biodegradable polymers. The primary emulsion is then mixed with a second aqueous solution to make a double emulsion of water-in-oil-in-water (W₁/O/W₂). The second aqueous solution comprises the active agent. The double emulsion is dried such that three-layer particles are obtained. Each of the three-layer particles comprises a second dosing layer, a second intervening layer and a third dosing layer, wherein the second dosing layer covers the second intervening layer, the second intervening layer covers the third dosing layer, wherein the second and third dosing layers comprise the active agent and the second intervening layer comprises the one or more biodegradable polymers. The three-layer particles are mixed with a third aqueous solution and a second organic phase to make a mixture. The third aqueous solution comprises the active agent and the second organic phase comprises the one or more biodegradable polymers. The mixture is dried so that five-layer particles are obtained. The five-layer particles comprise the active agent and the one or more biodegradable polymers, wherein each of the five-layer particles comprises a first dosing layer, a first intervening layer, the second dosing layer, the second intervening layer and the third dosing layer, wherein the first dosing layer covers the first intervening layer, the first intervening layer covers the second dosing layer, and the first intervening layer separates the first dosing layer from the second dosing layer, wherein the second dosing layer covers the second intervening layer, the second intervening layer covers the third dosing layer, and the second intervening layer separates the second dosing layer from the third dosing layer, wherein the first, second and third dosing layers comprise at least 90 wt % of the active agent and the first and second intervening layers comprise at least 90 wt % of the one or more biodegradable polymers, wherein each of the first, second and third dosing layers is soluble in an aqueous environment and each of the first and second intervening layers is hydrolytically degradable.

In another embodiment, for the at least three-dose or five-layer particles, the preparation method comprises mixing a first aqueous solution with a first organic phase to make a primary emulsion of water-in-oil (W₁/O). The first aqueous solution comprises the active agent and the first organic phase comprises one or more biodegradable polymers. The primary emulsion is mixed with a second aqueous solution to make a double emulsion of water-in-oil-in-water (W₁/O/W₂). The second aqueous solution comprises the active agent. The organic phase in the double emulsion is evaporated such that two-layer particles within the second aqueous solution are obtained. Each of the two-layer particles comprises a second intervening layer and a third dosing layer, the second intervening layer covers the third dosing layer, the third dosing layer comprises the active agent, and the second intervening layer comprises the one or more biodegradable polymers. The two-layer particles in the second aqueous solution are dried to make three-layer particles. Each of the three-layer particles comprises a second dosing layer, the second intervening layer and the third dosing layer, the second dosing layer covers the second intervening layer and comprises the active agent. The three-layer particles are mixed with a third aqueous solution and a second organic phase to make a mixture. The third aqueous solution comprises the active agent and the second organic phase comprises the one or more biodegradable polymers. The second organic phase in the mixture is evaporated such that four-layer particles within the third aqueous solution are obtained. Each of the four-layer particles comprises a first intervening layer, the second dosing layer, the second intervening layer and the third dosing layer, wherein the first intervening layer covers the second dosing layer and comprises the one or more biodegradable polymers. The four-layer particles within the third aqueous solution are dried, and five-layer particles are obtained. The five-layer particles comprise the active agent and the one or more biodegradable polymers, wherein each of the five-layer particles comprises a first dosing layer, the second dosing layer, the third dosing layer, the first intervening layer, and the second intervening layer, wherein the first dosing layer covers the first intervening layer, the first intervening layer covers the second dosing layer, and the first intervening layer separates the first dosing layer from the second dosing layer, wherein the second dosing layer covers the second intervening layer, the second intervening layer covers the third dosing layer, and the second intervening layer separates the second dosing layer from the third dosing layer, wherein the first, second and third dosing layers comprise at least 90 wt % of the active agent and the first and second intervening layers comprise at least 90 wt % of the one or more biodegradable polymers, wherein each of the first, second and third dosing layers is soluble in an aqueous environment and each of the first and second intervening layers is hydrolytically degradable.

The preparation method according to the present invention may further comprise storing the particles at a temperature of 20-40° C., 20-30° C. or 22-28° C. for a time period (e.g., at least 6 months, 1 year or 2 years, and at least 70%, 75%, 80%, 85%, 90%, 95% or 99% of the activity of the active agent remains after storage.

According to the at least three-dose particle preparation method, the first aqueous solution may further comprise an excipient; and the third aqueous solution may further comprise an excipient. The second aqueous may or may not comprise an excipient. The particles may further comprise an excipient in the first dosing layer, the second dosing layer, or a combination thereof.

The particles prepared by the preparation method of the present invention are provided. The prepared particles are dry.

The prepared particles may have an average particle size of 0.01-1,000 μm, 0.1-500 μm 0.1-400 μm, 10-400 μm, 10-100 μm or 15-100 μm. At least 70%, 75%, 80%, 85%, 90%, 95%, 99% or 100% of the prepared particles may have a particles size of 0.01-1,000 μm, 0.1-500 μm 0.1-400 μm, 10-400 μm, 10-100 μm or 15-100 μm.

The active reagent in the prepared particles may be an active pharmaceutical ingredient (API) for treating or preventing a disease or disorder in a subject. In one embodiment, the active agent is selected from the group consisting of a vaccine antigen, an antibody, an antibody-drug conjugate (ADC), a peptide, a protein, a pharmaceutical active drug molecule, an immuno-modulator, a hormone, a cell and a combination thereof. The vaccine antigen may be selected from the group consisting of a protein, a polysaccharide, an epitope, a DNA, a RNA, a virus (e.g., attenuated virus or engineered virus), a virus-like particle, and a combination thereof. Where the active agent is a vaccine antigen, the particles may further comprise an adjuvant.

The biodegradable polymers in the prepared particles may be selected from the group consisting of poly (lactic-co-glycolic acid) (PLGA), which may have a ratio of lactic acid to glycolic acid ranging from 1:9 to 9:1, poly (lactic acid) (PLA) and combinations thereof.

The first dosing layer in the prepared particles may further comprise a cellulose-based polymer, a stabilizer, an antioxidant, a buffer, a bulking agent, or a combination thereof. The second dosing layer in the prepared particles may further comprise a cellulose-based polymer, a stabilizer, an antioxidant, a buffer, a bulking agent, or a combination thereof. The cellulose-based polymer may be selected from the group consisting of poly (vinyl alcohol) (PVA), ethyl cellulose (EC), methyl cellulose (MC), hydroxy propyl methyl cellulose (HPMC), hypromellose acetate succinate (HPMCAS), alginate, chitosan and a combination thereof. The chitosan may be glycol chitosan, EDTA-chitosan or N-trimethyl chitosan. The stabilizer may be selected from the group consisting of a carbohydrate, a hydrolyzed gelatin, a surfactant, a buffer salt, a culture medium, an antibiotic and a combination thereof. The carbohydrate may be selected from the group consisting of sucrose, trehalose, lactose, dextrose, galactose, maltose, maltodextrin, dextrin, glycogen, starch, and a combination thereof. The surfactant may be selected from the group consisting of polysorbate 80, sorbitan monooleate, sorbitan laurate, sorbitan stearate, sorbitan tristearate, sorbitan sesquioleate, sorbitane trioleate, sorbitan monopalmitate, polyoxyethylenesorbitan monopalmitate, polyethylene glycol, sorbitan monostearate, polyoxyethylenesorbitan trioleate, polyoxyethylenesorbitan tristearate, D-tocopherol, and combinations thereof.

A composition comprising the particles of the present invention or the prepared particles of the present invention is provided. The composition further comprises a pharmaceutically acceptable excipient. The composition may be in an oral, injectable, inhalational (e.g., through mouth or nose), sublingual, buccal, transdermal rectal or vaginal delivery form. The composition may be in the form of capsules (e.g., enteric capsules), tablets (e.g., oral dissolving tablets, enteric coated tablets, sustained release tablets, or osmotic tablets), granules (e.g., coated granules, enteric coated granules, or sustained release granules) or suspensions of particles for oral delivery to gastrointestinal (GI) tract. The composition may be in the form of an orally dissolvable film (ODF) for buccal or sublingual delivery. The composition may be in the form of a transdermal patch for transdermal delivery. The composition may be in the form of a cream, ointment or gel for topical delivery. The composition may be in the form of a hydrogel matrix for subcutaneous or intramuscular injection. The composition may be in an injectable form for intravenous, subcutaneous or intramuscular delivery.

A medicament comprising the particles of the present invention or the prepared particles of the present invention is provided. The medicament further comprises a pharmaceutically acceptable excipient. The medicament may be in an oral, injectable, inhalational (e.g., through mouth or nose), sublingual, buccal, transdermal rectal or vaginal delivery form. The medicament may be in the form of capsules (e.g., enteric capsules), tablets (e.g., oral dissolving tablets, enteric coated tablets, sustained release tablets, or osmotic tablets), granules (e.g., coated granules, enteric coated granules, or sustained release granules) or suspensions of particles for oral delivery to gastrointestinal (GI) tract. The composition may be in the form of an orally dissolvable film (ODF) for buccal or sublingual delivery. The medicament may be in the form of a transdermal patch for transdermal delivery. The medicament may be in the form of a cream, ointment or gel for topical delivery. The medicament may be in the form of a hydrogel matrix for subcutaneous or intramuscular injection. The medicament may be in an injectable form for intravenous, subcutaneous or intramuscular delivery.

A method for preparing the medicament is provided. The medicament preparation method comprises mixing the particles with a pharmaceutically acceptable excipient.

A method for providing at least two doses of an active agent to a subject after a single administration of the active agent to the subject. The providing method comprises administering a composition of the present invention to the subject. The composition comprises the particles of the present invention. The particles comprise an effective amount of the active agent and one or more biodegradable polymers. Each particle comprises a first dosing layer, a second dosing layer and a first intervening layer. The first dosing layer covers the first intervening layer. The first intervening layer covers the second dosing layer. The first intervening layer separates the first dosing layer from the second dosing layer. The first and second dosing layers comprise at least 80 wt %, 85 wt %, 90 wt %, 95 wt %, 99 wt % or 100 wt % of the active agent. The first intervening layer comprises at least 80 wt %, 85 wt %, 90 wt %, 95 wt %, 99 wt % or 100 wt % of the one or more biodegradable polymers. The first and the second dosing layers are soluble in an aqueous environment. The first intervening layer is hydrolytically degradable. The providing method further comprises providing a first dose of an active agent from the first dosing layer in the particles in the subject during a first dosing time period after the administration step; and providing a second dose of the active agent from the second dosing layer in the particles in the subject during a second dosing time period after providing the first dose. A first intervening time period separates the first dosing time period from the second dosing time period. As a result, the subject receives at least two doses of the active agent after a single administration of the active agent to the subject. The subject may suffer a disease or disorder, and the first and second doses of the active agent may be effective for treating or preventing the disease or disorder in the subject.

The subject may have first dosing, second dosing and first intervening average bodily fluid concentrations of the active agent during the first dosing, second dosing and first intervening time periods, respectively, and the first intervening average bodily fluid concentration may be no more than 50% of each of the first and second dosing average serum concentrations. The bodily fluid may be blood.

According to the providing method, each of the particles may further comprise a third dosing layer and a second intervening layer. The second dosing layer may cover the second intervening layer while the second intervening layer may cover the third dosing layer. The second intervening layer may separate the second dosing layer from the third dosing layer. The first, second and third dosing layers may comprise at least 80 wt %, 85 wt %, 90 wt %, 95 wt %, 99 wt % or 100 wt % of the active agent. The first and second intervening layers may comprise at least 80 wt %, 85 wt %, 90 wt %, 95 wt %, 99 wt % or 100 wt % of the one or more biodegradable polymers. The third dosing layer may be soluble in an aqueous environment and the second intervening layer may be hydrolytically degradable. The providing method may further comprise releasing a third dose of the active agent from the third dosing layer in the particles in the subject within a third dosing time period after providing the second dose. A second intervening time period may separate the second dosing time period from the third dosing time period. As a result, the subject may receive at least three doses of the active agent after a single administration of the active agent to the subject. The subject may have first dosing, second dosing, third dosing, first intervening and second intervening average bodily fluid concentrations of the active agent during the first dosing, second dosing, third dosing, first intervening and second intervening time periods, respectively, and each of the first and second intervening average bodily fluid concentrations may be no more than 50% of each of the first, second and third dosing average serum concentrations. The bodily fluid may be blood. The subject may suffer a disease or disorder, and the first, second and third doses of the active agent may be effective for treating or preventing the disease or disorder in the subject.

According to the providing method, the first intervening time period may be longer than the first dosing time period. The second dosing time period may be longer than the first dosing time period. The particles may further comprise an excipient in the first dosing layer, the second dosing layer, or a combination thereof. The particles may be administered to the subject via oral, injectable, inhalational (e.g., through mouth or nose), sublingual, buccal, transdermal, rectal or vaginal delivery.

Where the active agent is a vaccine antigen (e.g., anthrax protective antigen PA83), the providing method may further comprise inducing an immune response to the antigen in the subject during the first and second dosing time periods. The immune response may be a T-cell response or a B-cell response. The particles may further comprise an adjuvant in the first dosing layer, the second dosing layer or a combination thereof.

### Example 1. Fabrication of Multi-Layered Vaccine Particles

The fabrication of multi-layered vaccine particles is divided into two main steps. The first step is formulation preparation using the double emulsion technique. The second step involves drying of the formulation (using techniques such as spray drying, lyophilization, etc.) to obtain dry particles.

In the first step, a vaccine antigen (PA83 in our example) with or without an adjuvant (QS-21 or aluminum hydroxide in our example) is prepared at an appropriate concentration in an aqueous (W₁) buffer solution. This aqueous solution could also contain polymers (such as HPMCAS), sugars (such as trehalose), stabilizers (such as gelatin), and/or other components to aid in stabilizing the antigen during subsequent steps. In addition, an organic phase (O) consists of polymers such as PLGA or PLA dissolved in dichloromethane (DCM) with surfactants (such as Tween 80). When coating the aqueous inner core with PLGA or PLA in DCM, this organic phase becomes the outer phase, and the resulting bi-phasic solution is emulsified using a sonicator. The sonication process is carried out in 4 repeating cycles, each cycle consisting of one minute of sonication, followed by 30 seconds of resting on ice. This primary water-in-oil (W₁/O) emulsion is further emulsified with an aqueous outer phase (W₂) containing surfactant (PVA) using the same sonication process to make a double emulsion (W₁/O/W₂).

In the second step, the double emulsion (W₁/O/W₂) is spray dried using a spray dryer with a two-fluid atomizer nozzle to expose the double emulsion to a drying gas. The feed rates of the drying gas (e.g., nitrogen) and the double emulsion are optimized. Inlet temperature ranges from 100 to 200° C., with a minimum of 58° C. for the outlet temperature. Following drying, multi-layer vaccine particles are collected in a cyclone separator and stored in vials at room temperature in a desiccator. Alternative drying techniques, such as lyophilization or solvent evaporation, can also be used to obtain the dry powder. The resulting multi-layer vaccine particles have a core containing the antigen covered by a polymer layer, which is covered by another layer containing the antigen.

### Example 2. Spray Drying Process and Stability of Vaccine Antigen and Adjuvant

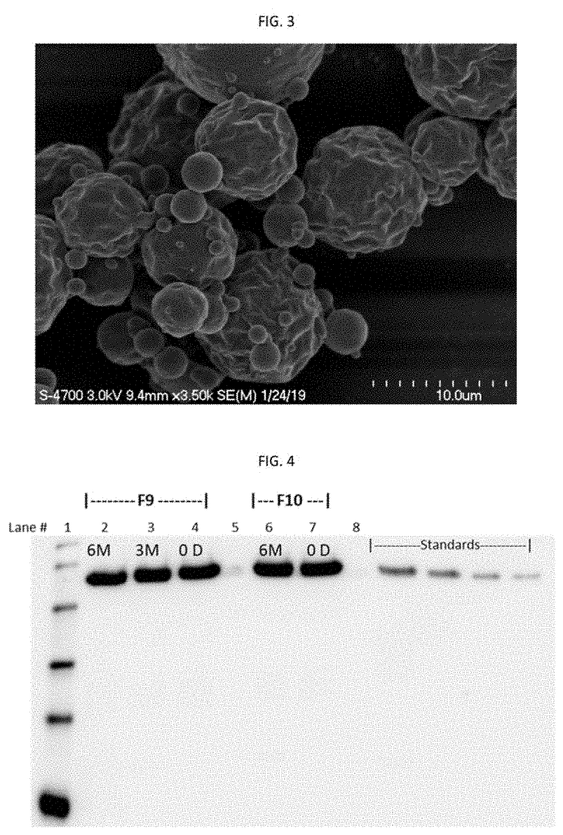

To establish the stability and appropriate concentrations of antigen and adjuvant to formulate using spray drying, we have assessed particles containing the vaccine antigen PA83 (deglycosylated anthrax protective antigen) and a saponin based adjuvant. The particles were spray dried at 110° C. and 160° C. to make formulations F9 and F10, respectively. The formulations contained other additives such as lyoprotectants (sugars), protein stabilizers, buffers, bulking agents, etc. FIG. 3 shows scanning electron microscope images of the spray dried particles containing the antigen and other components.

The formulations were stored at 25° C.±3° C. over a period of 6-months and their content and stability was analyzed using western blot analysis as shown in FIG. 4. There was no significant reduction in band intensity of samples stored for 6-months as compared to freshly spray dried samples, indicating that the antigen in the formulations was stable during storage. These results were also confirmed using a Bioanalyzer.

The saponin based adjuvant was also formulated and spray dried to produce formation F49. F49 comprises the saponin based adjuvant spray dried with additives. The summary from reverse phase analysis

(Table 1) confirms no significant difference in purity and concentration of adjuvant F49 compared to non-spray dried QS21 based adjuvant (FhCMB Adj). A hemolysis assay was also performed, which confirms bioactivity of this spray dried adjuvant formulation similar to the non-spray dried adjuvant.

TABLE 1
Reverse phase analysis of spray dried adjuvant formulation
Spray dried saponin based adjuvant

| Attribute | FhCMB Adj (Non-SD*) | F49 (SD*) |
|---|---|---|
| Purity, | 97.9% | 98.9% |
| Identity, FhCMB | Profile compares to | Profile compares to |
| Adj | reference material | reference material |
| Identity, | Profile compares to | Profile compares to |
| Phosphatidylcholine | reference material | reference material |
| Identity, Cholesterol | Profile compares to | Profile compares to |
| | reference material | reference material |
| Concentration, | 0.50 mg/mL | 0.43 mg/mL |
| FhCMB Adj | | |
| Concentration, | 0.18 mg/mL | 0.15 mg/mL |
| Phosphatidylcholine | | |
| Concentration, | 0.13 mg/mL | 0.11 mg/mL |
| Cholesterol | | |

*SD: Spray dried

These preliminary results demonstrate that the content and stability of the anthrax vaccine antigen and the adjuvant after spray drying are comparable to the non-spray dried formulations of the antigen and the adjuvant with no significant difference between the two. These were then progressed for the development of multi-layered formulations.

### Example 3. Multi-Layered Particles and their Characterization

We have generated a protein subunit vaccine with deglycosylated anthrax protective antigen PA83 formulations using spray drying technology to generate thermostable, recombinant vaccine particles. The polymers used are poly(lactic-co-glycolic acid) (PLGA) in 2 different ratios of LA:GA; PLGA 50:50, PLGA 75:25. The process parameters of spray drying (different drying temperatures—range from 100° C. to 220° C.) were optimized to generate stable powders. The size range of the multilayer particles is 15-100 μm. The particles have three layers, outer layer, middle or intervening layer and inner layer. When these particles are reconstituted in an aqueous buffer such as PBS, the outer layer is dissolved providing the first dose of the vaccine antigen. Western blot analysis confirms the presence of the vaccine antigen in the outer layer and the inner layer, but not the middle or intervening layer, of the multi-layered particles (FIG. 5).

The disruption/dissolution of the polymeric middle or intervening layer at a later point will lead to release of the second dose of the antigen from the inner layer.

### Example 4. Long-Term Stability Study

These spray dried multilayer particles prepared as described in Example 3 are being stored at 3 different temperatures, 25° C.±3° C., 40° C.±3° C. and 60° C.±3° C. and evaluated for stability and bioactivity of the antigen, PA83. At each time point, solid state analysis of the stored multilayered spray-dried PA83 formulations will assess appearance and surface morphology using Scanning Electron Microscopy (SEM), while moisture content, DSC and PXRD will be evaluated, and data will be compared to that for multi-layered spray-dried PA83 formulations at time zero (TO). Similarly, at each time point the spray-dried PA83 formulations will be reconstituted in Milli Q water (18Ω, 0.2 μm filtered) and evaluated for appearance, and presence and stability of PA83 by Western blot, SEC, SDS-PAGE, Bioanalyzer and RP-HPLC. Biological activity will be confirmed by TFA. These methods will evaluate product attributes over storage times that are likely to influence quality, safety and efficacy.

### Example 5. Immunization Mock Study

The multi-layered spray-dried PA83 particle formulations were investigated in a mouse immunogenicity mock study of four groups of mice (Table 2).

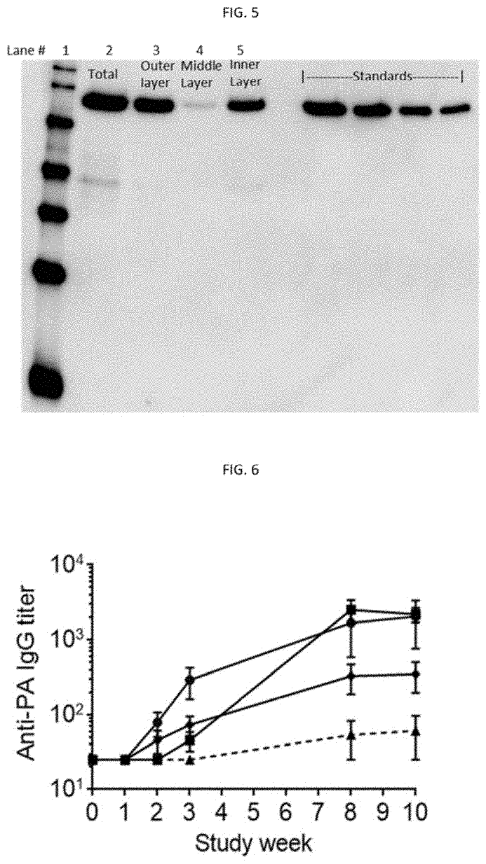

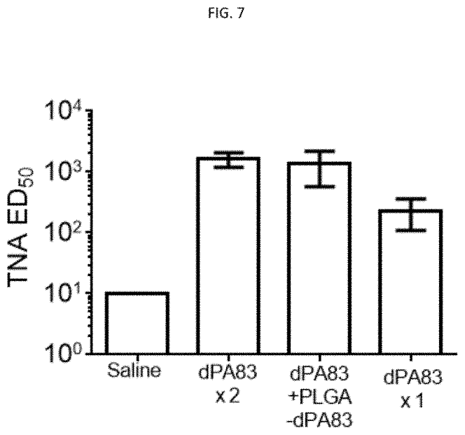

For group 1, saline was administered to each mouse on day 0 and day 21 (Group 1), providing no vaccine antigen PA83. Group 1 serves as a negative control (FIG. 6, triangles; FIG. 7, Saline).

For group 2, 5 μg of soluble antigen PA83 was administered to each mouse on day 0 and day 21, providing a prime dose of antigen PA83 on day 0 and a boost dose of antigen PA83 on day 21. Group 2 serves as a positive control with two doses of antigen PA83 on day 0 and day 21 (FIG. 6, squares; FIG. 7, PA83 x2).

For group 3, 5 μg of PA83 within a PLGA coating (PA83-PLGA) and 5 μg of soluble antigen PA83 were administered to each mouse on day 0 (Group 3), providing a prime dose from the soluble antigen PA83 on day 0 and a boost dose from the PA83-PLGA at a later time. Group 3 mimics three-layer particles having antigen PA83 in the outer and inner layers, but not the middle or intervening layer (FIG. 6, circles; FIG. 7, PA83+PLGA-dPA83).

For group 4, 10 μg of PA83 was administered to each mouse on day 0 (Group 4), providing a prime dose of on day 0. Group 4 serves as a positive control with a single dose of antigen PA83 on day 0 (FIG. 6, diamonds; FIG. 7, PA83 x1).

A QS21 based adjuvant was included in the prime dose on day 0 in all groups.

TABLE 2
Multilayer PA83 pilot immunogenicity study design

| | | | | Dose of | Dose of |
|---|---|---|---|---|---|
| | | | | Adjuvant | PA83 |
| | Mouse | Vaccine | | (μg) at | (μg) at |
| Group | # | Prime/boost | Route | day 0 and 21 | day 0 and 21 |
| 1 | 3 | Saline/Saline | IM | 10 + 0 | 0 + 0 |
| 2 | 5 | PA83/PA83 | IM | 10 + 0 | 5 + 5 |
| 3 | 5 | PA83 + | IM | 10 + 0 | 5 + (5) |
| | | PA83-PLGA | | | |
| 4 | 5 | PA83 | IM | 10 + 0 | 10 + 0 |

Serum samples were collected from all groups on days 0 (prior to injection), 7, 14, 21, 28, 42, and 63, and potentially later times. Serum samples from this study will be analyzed for anti-PA IgG and lethal toxin (LeTx) neutralizing (TN) antibody responses by ELISA and toxin neutralizing assay (TNA), respectively. Endpoint titers of anti-PA IgG and ED₅₀ values of TN antibody were analyzed statistically to understand the immunogenicity of multi-layered spray-dried formulations of PA83 and compared to those obtained from groups that received the comparator liquid formulation of PA83.

As shown in FIG. 6, group 3 receiving a single dose of PLGA-PA83 plus soluble dAP83 on day 0 (FIG. 6, circles) showed comparable anti-PA IgG to group 2 receiving two doses of soluble PA83 on day 0 and day 21 (FIG. 6, squares).

As shown in FIG. 7, group 3 receiving a single dose of PLGA-PA83 plus soluble PA83 on day 0 showed comparable Toxin Neutralizing Activity (TNA) to group 2 receiving two doses of soluble PA83 on day 0 and day 7. The results are consistent with the antibody titers shown in FIG. 6.

Although the invention is illustrated and described herein with reference to specific embodiments, the invention is not intended to be limited to the details shown. Rather, various modifications may be made in the details within the scope and range of equivalents of the claims without departing from the invention.

1. A method for providing a subject in need thereof at least two doses of an active agent after a single administration of the active agent to the subject, comprising
(a) administering a composition comprising particles to the subject, wherein the particles comprise the active agent and one or more biodegradable polymers, wherein each of the particles comprises a first dosing layer, a second dosing layer and a first intervening layer, wherein the first dosing layer covers the first intervening layer, the first intervening layer covers the second dosing layer, and the first intervening layer separates the first dosing layer from the second dosing layer, wherein the first and second dosing layers comprise at least 90 wt % of the active agent and the first intervening layer comprises at least 90 wt % of the one or more biodegradable polymers, wherein each of the first and the second dosing layers is soluble in an aqueous environment and the first intervening layer is hydrolytically degradable;
(b) providing a first dose of the active agent from the first dosing layer in the particles to the subject during a first dosing time period after step (a); and
(c) providing a second dose of the active agent from second dosing layer in the particles to the subject during a second dosing time period after step (b), wherein a first intervening time period separates the first dosing time period from the second dosing time period, whereby the subject receives at least two doses of the active agent after a single administration of the active agent to the subject.

2. The method of claim 1, wherein each of the particles further comprises a third dosing layer and a second intervening layer, wherein the second dosing layer covers the second intervening layer, the second intervening layer covers the third dosing layer, and the second intervening layer separates the second dosing layer from the third dosing layer, wherein the first, second and third dosing layers comprise at least 90 wt % of the active agent and the first and second intervening layers comprise at least 90 wt % of the one or more biodegradable polymers, wherein the third dosing layer is soluble in an aqueous environment and the second intervening layer is hydrolytically degradable, the method further comprising
(d) providing a third dose of the active agent from the third dosing layer in the particles to the subject within a third dosing time period after step (c), wherein a second intervening time period separates the second dosing time period from the third dosing time period, whereby the subject receives at least three doses of the active agent after a single administration of the active agent to the subject.

3-9. (canceled)

10. The method of claim 1, wherein the active agent is a vaccine antigen, the method further comprising inducing an immune response to the antigen in the subject during the first and second dosing time periods.

11-12. (canceled)

13. The method of claim 10, wherein the particles further comprise an adjuvant in the first dosing layer, the second dosing layer or a combination thereof.

14. A method for preparing three-layer particles for providing a subject in need thereof at least two doses of an active agent after a single administration of the active agent to the subject, comprising
(a) mixing a first aqueous solution with an organic phase, wherein the first aqueous solution comprises the active agent and the organic phase comprises one or more biodegradable polymers, whereby a primary emulsion of water-in-oil (W₁/O) is obtained;
(b) mixing the primary emulsion with a second aqueous solution, wherein the second aqueous solution comprises the active agent, whereby a double emulsion of water-in-oil-in-water (W₁/O/W₂) is obtained; and
(c) drying the double emulsion, whereby three-layer particles are obtained, wherein the three-layer particles comprise the active agent and the one or more biodegradable polymers, wherein each of the three-layer particles comprises a first dosing layer, a second dosing layer and a first intervening layer, wherein the first dosing layer covers the first intervening layer, the first intervening layer covers the second dosing layer, and the first intervening layer separates the first dosing layer from the second dosing layer, wherein the first and second dosing layers comprise at least 90 wt % of the active agent and the first intervening layer comprises at least 90 wt % of the one or more biodegradable polymers, wherein each of the first and the second dosing layers is soluble in an aqueous environment and the first intervening layer is hydrolytically degradable.

15. The method of claim 14, further comprising steps (d) and (e) in place of step (c):
(d) evaporating the organic phase in the double emulsion, whereby two-layer particles within the second aqueous solution are obtained, wherein each of the two-layer particles comprises an intervening layer and a second dosing layer, the intervening layer covers the second dosing layer, the second dosing layer comprises the active agent, and the intervening layer comprises the one or more biodegradable polymers; and
(e) drying the two-layer particles within the second aqueous solution, whereby three-layer particles are obtained.

16. The method claim 14, further comprising step (f)-(h) in place of step (c):
(f) drying the double emulsion, whereby three-layer particles are obtained, wherein each of the three-layer particles comprises a second dosing layer, a second intervening layer and a third dosing layer, wherein the second dosing layer covers the second intervening layer, the second intervening layer covers the third dosing layer, wherein the second and third dosing layers comprise the active agent and the second intervening layer comprises the one or more biodegradable polymers;
(g) mixing the three-layer particles with a third aqueous solution and a second organic phase, wherein the third aqueous solution comprises the active agent and the second organic phase comprises the one or more biodegradable polymers, whereby a mixture is prepared; and
(h) drying the mixture, whereby five-layer particles are obtained, wherein the five-layer particles comprise the active agent and the one or more biodegradable polymers, wherein each of the five-layer particles comprises a first dosing layer, a first intervening layer, the second dosing layer, the second intervening layer and the third dosing layer, wherein the first dosing layer covers the first intervening layer, the first intervening layer covers the second dosing layer, and the first intervening layer separates the first dosing layer from the second dosing layer, wherein the second dosing layer covers the second intervening layer, the second intervening layer covers the third dosing layer, and the second intervening layer separates the second dosing layer from the third dosing layer, wherein the first, second and third dosing layers comprise at least 90 wt % of the active agent and the first and second intervening layers comprise at least 90 wt % of the one or more biodegradable polymers, wherein each of the first, second and third dosing layers is soluble in an aqueous environment and each of the first and second intervening layers is hydrolytically degradable.

17. The method of claim 14, further comprising steps (i)-(m) in place of step (c):
(i) evaporating the organic phase in the double emulsion, whereby two-layer particles within the second aqueous solution are obtained, wherein each of the two-layer particles comprises a second intervening layer and a third dosing layer, the second intervening layer covers the third dosing layer, the third dosing layer comprises the active agent, and the second intervening layer comprises the one or more biodegradable polymers;
(j) drying the two-layer particles in the second aqueous solution, whereby three-layer particles are obtained, wherein each of the three-layer particles comprises a second dosing layer, the second intervening layer and the third dosing layer, the second dosing layer covers the second intervening layer and comprises the active agent;
(k) mixing the three-layer particles with a third aqueous solution and a second organic phase, wherein the third aqueous solution comprises the active agent and the second organic phase comprises the one or more biodegradable polymers, whereby a mixture is prepared;
(l) evaporating the second organic phase in the mixture, whereby four-layer particles within the third aqueous solution are obtained, wherein each of the four-layer particles comprises a first intervening layer, the second dosing layer, the second intervening layer and the third dosing layer, wherein the first intervening layer covers the second dosing layer and comprises the one or more biodegradable polymers; and
(m) drying the four-layer particles within the third aqueous solution, whereby five-layer particles are obtained, wherein the five-layer particles comprise the active agent and the one or more biodegradable polymers, wherein each of the five-layer particles comprises a first dosing layer, the second dosing layer, the third dosing layer, the first intervening layer, and the second intervening layer, wherein the first dosing layer covers the first intervening layer, the first intervening layer covers the second dosing layer, and the first intervening layer separates the first dosing layer from the second dosing layer, wherein the second dosing layer covers the second intervening layer, the second intervening layer covers the third dosing layer, and the second intervening layer separates the second dosing layer from the third dosing layer, wherein the first, second and third dosing layers comprise at least 90 wt % of the active agent and the first and second intervening layers comprise at least 90 wt % of the one or more biodegradable polymers, wherein each of the first, second and third dosing layers is soluble in an aqueous environment and each of the first and second intervening layers is hydrolytically degradable.

18-22. (canceled)

23. The method of claim 1, wherein the active agent is selected from the group consisting of a vaccine antigen, an antibody, an antibody-drug conjugate (ADC), a peptide, a protein, a pharmaceutical active drug molecule, an immuno-modulator, a hormone, a cell, and a combination thereof.

24-25. (canceled)

26. The method of claim 1, wherein the first dosing layer further comprises a cellulose-based polymer, a stabilizer, an antioxidant, a buffer, a bulking agent, or a combination thereof.

27. The method of claim 1, wherein the second dosing layer further comprises a cellulose-based polymer, a stabilizer, an antioxidant, a buffer, a bulking agent, or a combination thereof.

28-31. (canceled)

32. The method of claim 14, wherein the active agent is a vaccine antigen, and the particles further comprise an adjuvant.

33. (canceled)

34. Particles prepared according to the method of claim 14.

35. Particles for providing a subject in need thereof at least two doses of an active agent after a single administration of the active agent to the subject, comprising the active agent and one or more biodegradable polymers, wherein each of the particles comprises a first dosing layer, a second dosing layer and a first intervening layer, wherein the first dosing layer covers the first intervening layer, the first intervening layer covers the second dosing layer, and the first intervening layer separates the first dosing layer from the second dosing layer, wherein the first and second dosing layers comprise at least 90 wt % of the active agent and the first intervening layer comprises at least 90 wt % of the one or more biodegradable polymers, wherein each of the first and the second dosing layers is soluble in an aqueous environment and the first intervening layer is hydrolytically degradable.

36. (canceled)

37. The particles of claim 35 or 36, wherein the active reagent is selected from the group consisting of a vaccine antigen, an antibody, an antibody-drug conjugate (ADC), a peptide, a protein, a pharmaceutical active drug molecule, an immuno-modulator, a hormone, a cell, and a combination thereof.

38. The particles of claim 37, wherein the vaccine antigen is selected from the group consisting of a protein, a polysaccharide, an epitope, a DNA, a RNA, a virus, a virus-like particle, and a combination thereof.

39. (canceled)

40. The particles of claim 35, wherein the first dosing layer further comprises a cellulose-based polymer, a stabilizer, an antioxidant, a buffer, a bulking agent, or a combination thereof.

41. The particles of claim 35, wherein the second dosing layer further comprises a cellulose-based polymer, a stabilizer, an antioxidant, a buffer, a bulking agent, or a combination thereof.

42-45. (canceled)

46. The particles of claim 35, wherein the active agent is a vaccine antigen, the particles further comprising an adjuvant.

47. (canceled)

48. A composition comprising the particles of claim 35 and a pharmaceutically acceptable excipient.

49-55. (canceled)
